(19) United States
(12) Patent Application Publication
(10) Pub. No.: US 2020/0049726 A1
(43) Pub. Date: Feb. 13, 2020

(54) SAMPLING DEVICE AND QUALITY ASSURANCE OF SAMPLING PROCEDURE

(71) Applicant: PROVTAGAREN AB, Hässleholm (SE)

(72) Inventors: Daniel Ottosen, Lund (SE); Gunnar Skarping, Hässleholm (SE); Marianne Dalene, Hössleholm (SE)

(21) Appl. No.: 16/492,707

(22) PCT Filed: Mar. 9, 2018
(86) PCT No.: PCT/SE2018/050228

§ 371 Date: Sep. 10, 2019

(30) Foreign Application Priority Data
Mar. 10, 2017 (SE) 1750272-5

(51) Int. Cl. G01N 35/00; G01F 1/00; G01N 1/22; G01N 33/00; G01N 1/24

(52) U.S. Cl. CPC G01N 35/00871; G01F 1/00; G01N 1/2273; G01N 33/0004; G01N 2035/00881; G01N 2035/00752; G01N 2035/00762; G01N 2035/00772; G01N 2035/00782; G01N 1/24

(57) ABSTRACT

The invention relates to a fluid sampling assembly comprising a sampling device (1) for analysis of one or more substances in a fluid flow passing through the sampling device (1), a first device (3) comprising a flow meter for measuring the fluid flow, and a control unit (12) with communication means, wherein the control unit (12) is adapted to record information concerning the sampling, and to communicate the information to a memory (4, 7, 13). The invention further relates to a method for assuring quality of measurements using a sampling assembly.

### FIELD OF THE INVENTION

The present invention relates to a sampling device and method to provide quality assurance for the conduct of a sampling session performed with such a sampling device.

### BACKGROUND ART

The procedures of collection and evaluating compounds in environments without using direct reading instruments usually comprise the steps of using a sampling device, collecting one or several certain compounds to this sampling device, thereafter seal the sampling device and send it to a laboratory, regulatory body or other institution for evaluation. In order to properly evaluate the sampling device results, the institution needs information about sampling procedure. The current procedures vary between operators. An example of a procedure when sampling an air sample may be: 1) Writing down sampling device ID, 2) calibrate and start a gas pump to a certain flow rate, the gas pump connected to the sampling device, 3) writing down elapsed time and/or calculating total volume of air which have passed through the sampling device. 4) Send the sampling device and the written protocol to the laboratory which evaluates the sampling device.

There are numerous possible sources for errors in the present procedures for collection sampling device procedure information on a sampling session log protocol. The operator may accidentally write down the wrong sampling device ID. Due to time constraints or lack of information, the operator would not provide sufficient information on the log protocol. The operator misses or neglects important information, or calculates results erroneously. For an operator handling several sampling sessions simultaneously, there is a risk for mixing different sampling devices with different log protocols. The calibration may be performed poorly, affecting accuracy of flow rate throughout the measurement. When air flows through the sampling device, there is an atmospheric pressure difference over the sampling device (backpressure), which may affect flow rate. Even though the backpressure over the sampling device has been taken into account for when performing calibration, the backpressure often changes over the sampling device during a sampling session, and depending on how stable the gas pump can maintain a flow when backpressure and other ambient conditions changes, the flow rate may change during the sampling session. Many setups, especially carried gas pumps and sampling devices add extra errors due to movement, e.g. tubes connected between gas pump and sampling device that has been choked, or tubes that has been disconnected between gas pump and sampling device. A gas pump may terminate its flow prematurely due to battery failure or other reasons. A sampling session may run for a full work day, e.g. 8-12 hours. Most gas pumps used in air sampling induces a pulsating flow, whereas not only average flow but also pulsation amplitude and frequency may affect sampling device result considerably.

All errors that may be derived to human errors, mistakes and neglects of sufficient information as well as the time consuming procedure to summarize a sampling session into a log protocol, proves that there is a need for better automatic generation of the log protocols, this for eliminating human errors as well as reduce time needed for operators.

### SUMMARY OF THE INVENTION

An object of the present invention is to eliminate the above-mentioned problems and provide a fluid sampling assembly with which the quality of a fluid sampling measurement may be assured. According to the invention, this object is achieved by a fluid sampling assembly comprising a sampling device for analysis of one or more substances in a fluid flow passing through the sampling device, a first device comprising a flow meter for measuring the fluid flow, and a control unit with communication means, wherein the control unit is adapted to record information concerning the sampling, and to communicate the information to a memory. Such a sampling device may store information regarding the measurement that may later be checked by the laboratory when the sampling device is evaluated and processed to obtain its measurement results. It is thus possible to check that requirements of the measurement such as flow rate during sampling and sampling time are met. The quality of the measurement is thereby assured as far as possible. Human errors can be detected and erroneous measurements can be discarded.

The sampling device may further comprise identification means, such as a unique ID. The unique ID can be used for linking a set of stored data for a sampling session with a particular sampling device. This opens the possibility of having a memory that is located in network like a cloud solution with access over the internet.

The identification means may be a Near Field Communication (NFC) tag, a QR tag, a barcode, an RFID tag, regular text, a number or numbers, or any graphical symbol.

The memory may however instead or also be located on the sampling device, and the memory or the sampling device may further comprise communications means. The communication means is then preferably a low power induction driven communication means like an inductively driven wireless circuit as an NFC tag or a low power communication means like a Bluetooth device or the like.

The memory may also provide the unique ID when the memory is an NFC tag.

The recorded information may be e.g. time and date for the sampling, flow through the sampling device, the sampling device unique identification, raw flow data from the sampling procedure, processed flow data from the sampling procedure, the ambient pressure, the ambient temperature, back pressure from the sampling device, accelerometer data from the first device or the fluid assembly, and/or concentration.

It is very important the ID check is correctly performed as a sampling operator sometimes brings a number of different sampling devices. To be sure that the identified sampling device is also the sampling device that was used for the measurement, the ID check may be performed a second time at the end of the measurement. If the ID tag is an inductively driven wireless circuit as an NFC tag, the first device may do the ID check automatically during or at the end of the sampling session. If the ID tag has to be read by the first device without active two-way communication, e.g. when an optical check is performed, the operator may be reminded at the end of the measurement session to perform a second ID-check to verify that the ID of the sampling device that was used during for the fluid sampling.

According to one embodiment the first device further comprises a pump, i.e. the pump and flow meter are integrated in one and the same device.

The fluid assembly may further comprise a biometric sensor arranged to communicate with the control unit. The biometric sensor may be a fingerprint reader, an eye scanner, face recognition or any similar biometric identification device. Having a biometric sensor opens the possibility of checking the identification of the operator and e.g. check that the operator is an authorized to do a measurement.

It is not uncommon that air sampling is conducted for air evaluation to determine if the air is healthy to breath making the integrity of the sampling results very important. In that aspect one important factor is the reliability of the operator. An authorized operator minimizes the risk of manipulation of the measurement. If manipulation of measurements is discovered, a biometric sensor will with certainty prove who was the operator for a certain fluid sampling session.

The fluid sampling assembly may further comprise a second device communicating with the first device, said second device comprising features that may thereby be omitted on the first device. The second device could e.g. be a smart phone or a tablet computer device functioning as a user interphase for the operator for controlling the first device. The second device could also contain the biometric sensor, connection to a network as the Internet and other conventional features that may be used by the first device so that they may be omitted on the first device.

A further aspect of the invention involves a method for assuring quality of measurements of a fluid sampling assembly, the fluid sampling assembly comprising a sampling device for analysis of one or more substances in a fluid flow passing through the sampling device, a first device comprising a flow meter for measuring the fluid flow, and a control unit with wireless communication means, wherein the method comprises the steps of sampling the one or more substances in a fluid flow passing through the sampling device, recording information concerning for the sampling, and communicating the information to a memory.

The method may further comprise the step of identifying a unique identification of the sampling device. The step of recording information may further comprise preparing information in a log file. The method may further comprise the step of communicating information to the memory. The method may further comprise write protecting information in the memory so that the information cannot be manipulated after the measurement data has been written to the memory.

The method may further comprise the step of checking if the sampling device has previously been used, by comparing first logged use of the sampling device with current time or by checking if or when a digital sealing was broken.

An alternative method for checking if the sampling device has previously been used is to use a seal and check if that seal has been broken. Such a seal is possible to implement using a NFC seal or any similar digital hardware seal. The seal could however also be a traditional seal using a label that is read by the operator before it is broken and thereby destroyed as seal.

The time stamp for the measurement may be important as some sampling devices have an expiration time. By storing the time of measurement in the memory, it can later on be checked that the sampling device had not expired when the measurement was performed.

It should be noted that the inventive method may incorporate any of the features described above in association of the inventive device and has the same corresponding advantages.

Generally, all terms used in the claims are to be interpreted according to their ordinary meaning in the technical field, unless explicitly defined otherwise herein. All references to “a/an/the [element, device, component, means, step, etc.]” are to be interpreted openly as referring to at least one instance of the element, device, component, means, step, etc., unless explicitly stated otherwise. The steps of any method disclosed herein do not have to be performed in the exact order disclosed, unless explicitly stated.

As used herein, the term “comprising” and variations of that term are not intended to exclude other additives, components, integers or steps.

### BRIEF DESCRIPTION OF THE DRAWINGS

The above objects, as well as additional objects, features and advantages of the present invention, will be more fully appreciated by reference to the following illustrative and non-limiting detailed description of preferred embodiments of the present invention, when taken in conjunction with the accompanying drawings, wherein:

FIG. 1 schematically shows how to automatically retrieve unique identification of a sampling device and tie it to the memory integrated in the device to be used for logging sampling session information,

FIG. 2 schematically shows how to automatically provide information about the sampling session stored in the memory integrated in the device to the instance which will evaluate the sampling results, and

FIG. 3 schematically shows how to automatically provide the information about the sampling session to the instance which will evaluate the sampling results by storing information on a remote memory on a network.

### DETAILED DESCRIPTION OF PREFERRED EMBODIMENTS OF THE INVENTION

Some expressions used throughout this application are defined as follows.

The expression “sampling device” refers to a device collecting compounds for evaluating an environmental condition, e.g. the concentration of certain hazardous compounds in environmental air.

The expression “sampling procedure” refers to the procedure of collecting compounds into the sampling device.

The expression “sampling session” refers to the timespan over which this procedure is performed.

The expression “sample” refers to the sampling device and the compounds it has been exposed to during the sampling session. The sample is the item sent to an instance (e.g. a laboratory) for evaluation.

The expression “operator” refers to the person responsible of the quality of the sampling procedure and collecting information of the sampling procedure. This is usually an occupational hygienist.

An example of a situation where the method the present invention may be used is when sampling hazardous compounds in environmental air, using an air pump 3 connected to a sampling device 1, enabling the air pump 3 to induce a flow of environmental air through the sampling device during a certain time, this previously referred to as the sampling procedure. The sampling device and air pump may be attached to a person (usually not the operator, but a person working in present environment), the sampling device following the person, being exposed to the same environment as the person. After completing this procedure, the sampling device will be sealed and sent to e.g. a laboratory 9 for evaluation. Parameters in this example that are of interest in concluding an evaluation can be, but not limited to: sampling duration, date and time, average air flow rate, ambient temperature, ambient pressure, characteristics of air flow rate such as pulsations and deviations of average air flow rate, delta pressure over sampling device. The log file 6 containing such information must be tied to the sampling device 1 exposed to that exact sampling procedure described by the log file 6.

The purpose of the present invention is to improve the collection of information of how a sampling session of a certain sample has been conducted, and to provide this information to the instance which will evaluate the sample, while minimizing the manual workflow needed for doing this, and thus both reducing the risk of human errors and reduce time required to gather, summarize, and provide sufficient data of sampling procedure.

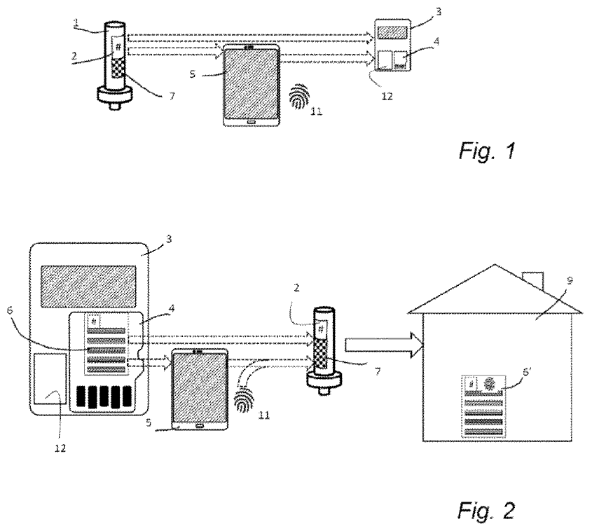

An exemplary embodiment will now be described with reference to FIGS. 1 and 2. A sampling device 1 is provided to gather a certain sample of e.g. environmental air is equipped provided with a unique identification, that can be automatically read, e.g. an RFID tag, a QR or barcode tag, or a Near-Field-Communication (NFC)-tag as shown as 2 in FIG. 1. This identification can be either read-only (as in the RFID or QR example), or also have a memory 7 that is able to be programmed multiple times (as for the NFC example). By utilizing a first device 3 that automatically reads the identification and provides it to a log file on a memory 4, the log file becomes tied to the unique sampling device 1. The identification could also be read by an optional second device 5, communicatively connected to the first device, where the second device transfers the identification information to the first device. The second device could be a smartphone or tablet device, using readily available means on such devices for reading the identification tags.

To a further aspect, the sampling device may contain data which can be read automatically in a similar manner as previously described, the data containing information how the sampling procedure should be performed, e.g. sampling flow rate and duration. The first device may be able to automatically adjust settings thereafter, e.g. a flow control set point and a timeout where the duration specified in the data has elapsed.

The corresponding prior art workflow is having an operator enumerating the sampling device, writing down the enumeration on a paper protocol or manually to an electronic protocol. This current procedure is often a compromise between consuming time for the operator and performing a sufficiently reliable enumeration.

During the sampling session, the first device performs an automatic collection of information continuously throughout the sampling procedure, and appends it to the log file 6, 6′. A sampling session may take several hours to complete. With the present invention, the operator can by evaluating the collected information assure the quality of the sampling procedure. To a further aspect, the first device 3 may detect an error in sampling procedure and signal the error to the operator. The error may either be signaled optically, by a sound signal or transferred via a wireless connection to the operator, thus reducing the requirement of the operator to closely monitor the sampling procedure throughout the sampling session.

When a sampling session is completed, either automatically due to the pre-programmed timeout or manually by the operator, the control unit 12 of the first device 3 summarizes the log file 6.

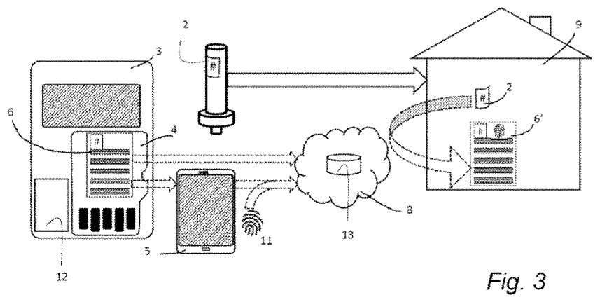

Should the sampling device used throughout the sampling session be equipped with a programmable memory 7, the log file 6 or a summary of the log file is programmed to the memory, either by using the first device 3 as programmer, or sending the log file to a secondary device 5, and then using the secondary device as programmer. The secondary device may also be used to transfer the log file to a database 13 available via a network 8, as e.g. the World Wide Web, as illustrated in FIG. 3. The laboratory 9 which will evaluate the results of the sampling device can use the unique identifier 2 of the sampling device 1 in order to access the log file 6 from the database, thus receiving all information about the sampling procedure of the sampling device in a local copy of the log file 6′ at the laboratory 9.

On a further aspect, the operator may add his/her signature on the log file prior to program the log file to the memory and/or transfer the log file to the database. The signature may be a biometric signature 11 added by using biometric sensors readily available on smartphones and tablets.

It is understood that other variations in the present invention are contemplated and in some instances, some features of the invention can be employed without a corresponding use of other features. Accordingly, it is appropriate that the appended claims be construed broadly in a manner consistent with the scope of the invention.

1. A fluid sampling assembly comprising:
a sampling device for analysis of one or more substances in a fluid flow passing through the sampling device,
a first device comprising a flow meter for measuring the fluid flow, and
a control unit with communication means,
wherein the control unit is adapted to record information concerning the sampling, and to communicate the information to a memory.

2. The fluid sampling assembly according to claim 1, wherein the sampling device further comprises identification means.

3. The fluid sampling assembly according to claim 1, wherein the memory is located on the sampling device, and the memory or the sampling device comprises communications means.

4. The fluid sampling assembly according to claim 1, further comprising a second device communicating with the first device, said second device comprising features that may thereby be omitted on the first device.

5. The fluid sampling assembly according to claim 1, wherein the memory is an inductively driven wireless circuit tag.

6. The fluid sampling assembly according to claim 2, wherein the memory is located in network.

7. The fluid sampling assembly according to claim 2, wherein the identification means comprises at least one of an inductively driven wireless circuit, NFC, QR, barcode, RFID, text, number, symbol, and electronic chip.

8. The fluid sampling assembly according to claim 1, wherein the record information is information comprising at least one of time, flow, sampling device identification, processed flow data from the sampling, ambient pressure, temperature, back pressure, accelerometer data, concentration, deviation event, error reports, and event reports.

9. The fluid sampling assembly according to claim 1, wherein the first device further comprises a pump.

10. The fluid sampling assembly according to claim 1, further comprises a biometric sensor arranged to communicate with the control unit.

11. A method for assuring quality and validity of measurements of a fluid sampling assembly, the fluid sampling assembly comprising:
a sampling device for analysis of one or more substances in a fluid flow passing through the sampling device,
a first device comprising a flow meter for measuring the fluid flow, and
a control unit with wireless communication means,
wherein the method comprises:
sampling the one or more substances in the fluid flow passing through the sampling device,
recording information concerning the sampling, and
communicating the information to a memory.

12. The method according to claim 10, further comprising identifying a unique identification of the sampling device.

13. The method according to claim 10, where recording information comprises preparing the information in a log file.

14. The method according to claim 10, further comprising communicating information to the memory.

15. The method according to claim 10, further comprising write protecting the information in the memory.

16. The method according to claim 10, further comprising checking if the sampling device has previously been used, by comparing first recorded use of the sampling device with current time or by checking if or when a digital sealing was broken.
